(19) United States
(12) Patent Application Publication
(10) Pub. No.: US 2007/0202522 A1
(43) Pub. Date: Aug. 30, 2007

(54) ISOTHERMAL SCREENING OF TUMOR CELL RELATED NUCLEIC ACIDS

(76) Inventor: Frank G. Salinas, Wheaton, IL (US)

Correspondence Address: MCANDREWS HELD & MALLOY, LTD, 500 WEST MADISON STREET, SUITE 3400, CHICAGO, IL, 60661, US

(21) Appl. No.: 11/679,718

(22) Filed: Feb. 27, 2007

Related U.S. Application Data
(60) Provisional application No. 60/776,988, filed on Feb. 27, 2006.

(51) Int. Cl. C12Q 1/68; C12Q 1/48; C12N 9/22

(52) U.S. Cl. 435/6; 435/15; 435/199

(57) ABSTRACT

The presently described technology relates generally to the art of molecular diagnostics and more particularly to point-of-care diagnostic methods and materials. The diagnostic methods and materials of the presently described technology are suitable for a variety of uses including but not limited to the bedside or field diagnosis of infectious or noninfectious diseases.

### RELATED APPLICATIONS

The present application is related to and claims priority from U.S. Provisional Patent Application Ser. No. 60/776,988, filed Feb. 27, 2006, the contents of which are hereby incorporated herein by reference in their entirety. Additionally, all cited references in the present application are hereby incorporated by reference in their entirety.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The presently described technology relates generally to the art of molecular diagnostics and more particularly to point-of-care diagnostic methods and materials. The diagnostic methods and materials of the presently described technology are suitable for a variety of uses including but not limited to the bedside or field diagnosis of infectious or noninfectious diseases. In particular, the presently described technology relates to the methods and materials for the detection of tumor cell related target sequences in a test sample.

Studies have suggested that the presence of epithelial cells in the hematopoietic system indicates the spread of cancer from a localized area to other parts of the body (also known as metastisis). This discovery is important since metastisis is diagnostic of certain stages of cancer, and decisions concerning the proper treatment of a cancer patient are largely dependent upon properly characterizing the stage of the disease. In particular, treatment of patients having localized cancer can be vastly different from treatment of patients in metastatic stages of cancer.

Early efforts to detect the spread of cancer by detecting epithelial cells in the hematopoietic system included immunocytological assay procedures. Unfortunately, these methods are largely inaccurate because antibodies used in these assays, and ostensibly specific for epithelial cells, demonstrate crossreactivity for cells normally found in the hematopoietic system. Hence, “normal hematopoietic cells” are sometimes detected in the absence of metastatic cells and therefore, false positive results can be obtained according to these assay procedures. Additionally, immunocytological assays lack sensitivity and can produce false negative results when low levels of epithelial cells are actually present in the hematopoietic system. Accordingly, early stages of metastatic cancer can be misdiagnosed using immunocytological asays.

Nucleic acid amplification based assays for detecting epithelial cells in the blood stream have provided significant advantages over immunocytological assay methods for detecting early stages of metastatic cancer. Most of these assays target a nucleic acid sequence encoding cytokeratin 19 (CK19), a protein found on the surface of epithelial cells. However, psuedogenes (comprising a nucleic acid sequence that closely mimics the gene for CK19) are present in the human genome. Thus, one challenge facing those developing amplification assays to detect a CK19 target sequence is to design assays that amplify and detect a sequence from the CK19 gene but not the closely related pseudogene.

### BRIEF SUMMARY OF THE INVENTION

One object of the present invention is to provide a molecular diagnostic system comprising methods and materials for the isothermal screening and detection of nucleic acids. Still another object of the present invention is to provide a molecular diagnostic system comprising methods and reagents for the isothermal screening and detection of nucleic acids associated with but not limited to disease, disease predisposition, disease causative agents, and any combination or derivative thereof. A further object of the present invention is to provide a molecular diagnostic system comprising methods and materials for the isothermal screening and detection of nucleic acids associated with tumor cells.

One or more of the preceding objects, or one or more other objects which will become plain upon consideration of the present specification, are satisfied by the invention described herein.

One aspect of the invention, which satisfies one or more of the above objects, is a test kit having reagents for the isothermal detection of nucleic acids associated with but not limited to disease, disease predisposition, disease causative agents, and any combination or derivative thereof. Another aspect of the invention is a test kit comprising: a strand transferase component; a polymerase component; and one or more primers and/or probes complementary to one or more nucleic acids associated with but not limited to disease, disease predisposition, disease causative agents, and any combination or derivative thereof. One preferred aspect of the present invention is a test kit comprising: a reverse transcriptase, a strand transferase component; a DNA dependent DNA polymerase component; and one or more primers and/or probes complementary to one or more nucleic acids associated with tumor cells.

### BRIEF DESCRIPTION OF SEVERAL VIEWS OF THE FIGURES

FIG. 1 is a schematic view of one aspect of the isothermal DNA amplification system of the present invention employing one primer complementary to a target nucleic acid, a strand transferase, and a polymerase.

FIG. 2 is a schematic view of another aspect of the isothermal DNA amplification system of the present invention employing two primers complementary to opposite strands and flanking a target nucleic acid, a strand transferase and a polymerase.

### DETAILED DESCRIPTION OF THE INVENTION

The present invention provides methods and materials for the isothermal screening and detection of nucleic acids associated with but not limited to disease, disease predisposition, disease causative agents, and any combination or derivative thereof. As used herein, and without limitation, nucleic acid generally includes any size DNA, RNA, DNA/RNA hybrid, or analog thereof. The nucleic acid can be single stranded, double stranded, or a combination of single and double stranded. As used herein, and without limitation, disease generally includes an impairment of the normal state of the living animal or plant body or one of its parts that interrupts or modifies the performance of the vital functions, is typically manifested by distinguishing signs and symptoms, and is a response to environmental factors (as malnutrition, industrial hazards, or climate), to specific infective agents (as parasites, bacteria, or viruses), to inherent defects of the organism (as genetic anomalies), or to combinations or derivatives of these factors.

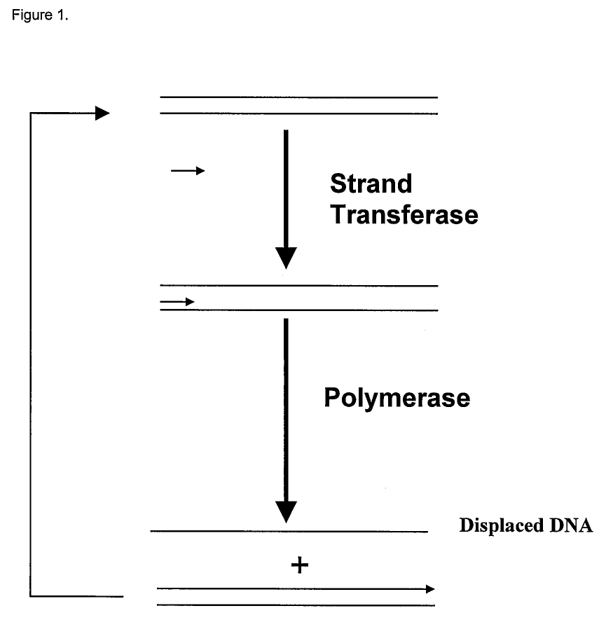

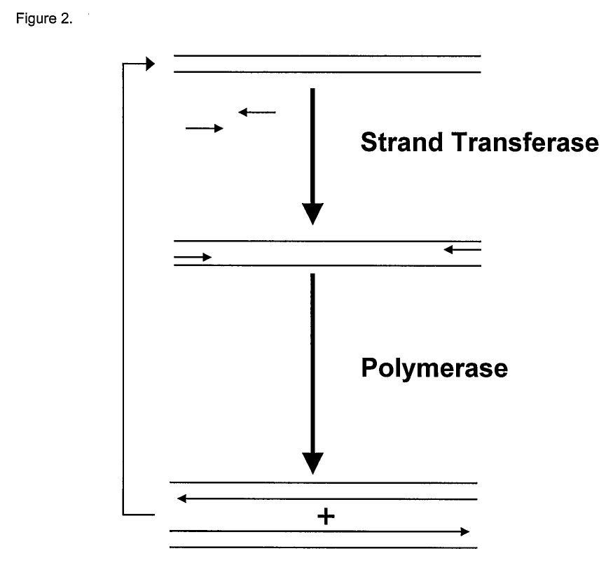

One aspect of the present invention includes methods and materials for the quantitative or qualitative isothermal screening and detection of one or more target nucleic acids of interest. This aspect of the present invention comprises contacting the target nucleic acid with at least one nucleic acid primer having complementarity to the target nucleic acid, a strand transferase, and a polymerase. The strand transferase catalyzes the homologous pairing of the at least one primer to a specific location on the target nucleic acid to form a primer-template junction that is acted upon by the polymerase to replicate and amplify the target nucleic acid (FIG. 1). In one preferred embodiment, the target nucleic acid is contacted with two primers complementary to opposite strands and flanking said target nucleic acid, in the presence of a strand transferase and a polymerase (FIG. 2). In certain aspects of the present invention, the isothermal amplification of the nucleic acid is performed as describe in U.S. Pat. No. 6,929,915, Methods for Nucleic Acid Manipulation. This reference is herein incorporated by reference.

As used herein without limitation, a strand transferase generally is a catalyst for the identification and base pairing of homologous sequences between nucleic acids, a process also known as homologous pairing or strand exchange. Bianco et al provides a general discussion of strand transferases in “DNA strand exchange proteins: a biochemical and physical comparison” at Front Biosci. Jun. 17, 1998; 3:D570-603. This reference is herein incorporated by reference. Strand transferases can be derived from either a prokaryotic system or an eukaryotic system, including but not limited to yeast, bacteria, and bacteriophages such as T4 and T7. For example West discusses eukaryotic strand transferases in Recombination genes and proteins“in Curr Opin Genet Dev. April 1994; 4(2):221-8. This reference is herein incorporated by reference. Radding discussed the recA strand exchange protein in “Helical RecA nucleoprotein filaments mediate homologous pairing and strand exchange” at Biochim Biophys Acta. Jul. 7, 1989; 1008(2):131-45. This reference is herein incorporated by reference. Also, the UvsX strand transferase was described by Kodadek et al., The mechanism of homologous DNA strand exchange catalyzed by the bacteriophage T4 uvsX and gene 32 proteins” JBC Jul. 5, 1988; 263(19):9427-36. This reference is herein incorporated by reference. Yonesaki discusses T4 homologous recombination in “Recombination apparatus of T4 phage” at Adv Biophys. 1995;31:3-22. This reference is herein incorporated by reference. Also, Salinas et. al have discussed the homology dependence of UvsX catalyzed strand exchange in “Homology dependence of UvsX protein-catalyzed joint molecule formation” at J Biol Chem. Mar. 10, 1995;270(10):5181-6. This reference is herein incorporated by reference. Exemplar strand transferase proteins include but are not limited to the eukaryotic Rad51 protein, the bacterial recA protein, the bacterial phage T4 UvsX protein, the bacteriophage T7 gene 2.5 or any protein fragment, derivative, or homolog thereof, including proteins found in nature and those engineered or modified using recombinant DNA technology. Kong et. al has discussed T7 strand exchange in “Role of the bacteriophage T7 and T4 single-stranded DNA-binding proteins in the formation of joint molecules and DNA helicase-catalyzed polar branch migration.” J Biol Chem. Mar. 28, 1997;272(13):8380-7. This reference is herein incorporated by reference.

Strand transferases generally operate by first binding single stranded regions of DNA to form a nucleoprotein filament generally referred to as the presynaptic filament. The presynaptic filament then binds a target nucleic acid and performs a search for homology that once complete results in the formation of a joint molecule or D-loop. Strand transferases generally have accessory protein factors that augment or modify their activity. For example, strand transferases generally have accessory protein factors that effect the formation and/or stability of the presynaptic filament under varying conditions, including for example buffer conditions and/or the presence of other proteins competing to bind regions of single-stranded nucleic acid. Exemplar strand transferase accessory proteins include but are not limited to the bacteriophage T4 UvsX accessory protein UvsY, the E. coli RecA accessory proteins RecFOR, the yeast and human Rad51 accessory protein Rad52, and any protein fragment, derivative, or homolog thereof, including proteins found in nature and those engineered or modified using recombinant DNA technology.

As used herein without limitation, a polymerase generally is any of several enzymes, such as DNA polymerase, RNA polymerase, or reverse transcriptase, that catalyze the formation of nucleic acid from precursor substances in the presence of preexisting nucleic acid acting as a template. The polymerase of the present invention can be derived from a eukaryotic or a prokaryotic system. For example the polymerase can be derived from a bacterium such as E.coli, a bacteriophage such as bacteriophage T4 or bacteriophage T7, a eukaryotic organism such as yeast or human, a virus, or any protein fragment, derivative, or homolog thereof, including proteins found in nature and those engineered or modified using recombinant DNA technology. Exemplar polymerases include but are not limited to the bacteriophage T4 gene product 43 protein, and any mutants or derivatives of the gene 43 protein including but not limited to the exonuclease deficient 43 exo⁻ polymerase. Benkovic et. al discusses replisome mediated DNA replication in “Replisome Mediated DNA Replication” at Annu Rev Biochem. 2001;70:181-208. This reference is herein incorporated by reference.

Polymerases generally have accessory protein factors that augment or modify their activity. Exemplar polymerase accessory factors include but are not limited to clamp proteins and clamp loader proteins. Clamp proteins generally have affinity and/or a topological link to both the polymerase and the nucleic acid being acted upon by said polymerase, thereby forming a stable link between polymerase and nucleic acid, the result of which is the formation of a stable polymerase nucleic acid complex having high processivity Clamp loader proteins facilitate the assembly of a clamp protein onto a nucleic acid and can also facilitate and mediate a concomitant or subsequent interaction with the polymerase. As used herein in connection with certain aspects and embodiments of the invention, the term holoenzyme generally regards a polymerase-clamp complex.

Polymerase accessory factors can be derived from a bacterium such as E.coli, a bacteriophage such as bacteriophage T4 or bacteriophage T7, a eukaryotic organism such as yeast or human, a virus, or any protein fragment, derivative, or homolog thereof, including proteins found in nature and those engineered or modified using recombinant DNA technology. Exemplar clamp proteins include but are not limited to the bacteriophage T4 gene product 45 protein, and any mutants or derivatives of the T4 gene product 45 protein. Trakselis et discuss the T4 polymerase holoenzyme in Creating a dynamic picture of the sliding clamp during T4 DNA polymerase holoenzyme assembly by using fluorescence resonance energy transfer” at Proc Natl Acad Sci U S A. Jul. 17, 2001;98(15):8368-75. This reference is herein incorporated by reference.

In certain embodiments of the present invention, the quantitative or qualitative isothermal screening and detection of one or more target nucleic acids of interest is performed in the presence of a single stranded nucleic acid binding protein (SSB). SSB's used pursuant to the present invention can be derived from a bacterium such as E.coli, a bacteriophage such as bacteriophage T4 or bacteriophage T7, a eukaryotic organism such as yeast or human, or any protein fragment, derivative, or homolog thereof, including proteins found in nature and those engineered or modified using recombinant DNA technology. Exemplar SSB's include but are not limited to the E.coli SSB protein, the bacteriophage T4 gene product 32 protein, the bacteriophage T7 gene product 2.5 protein, and the yeast or human RPA protein, or any mutants or derivatives thereof.

In certain embodiments of the present invention, the quantitative or qualitative isothermal screening and detection of one or more target nucleic acids of interest is performed in the presence of a helicase, preferably a DNA helicase. The helicase can be derived from a prokaryote or a eukaryote. For example, the DNA helicase can be from a bacterium such as E. coli., a bacteriophage such as bacteriophage T4 or bacteriophage T7, a yeast, or human. Exemplar helicases include but are not limited to the bacteriophage T4 gene product 41, the bacteriophage T4 dda protein, the bacteriophage T7 gene 4 protein, the E.coli UvrD protein, and any mutants or derivatives thereof. For example, Salinas and Kodadek have discussed the role of DNA helicases during strand homologous recombination in “Phage T4 homologous strand exchange: a DNA helicase, not the strand transferase, drives polar branch migration.” Cell Jul. 14, 1995;82(1):111-9. This reference is herein incorporated by reference. Also, Salinas and Benkovic have discussed the role of DNA helicases in bacteriophage T4 replication in “Characterization of bacteriophage T4-coordinated leading- and lagging-strand synthesis on a minicircle substrate.” Proc Natl Acad Sci U S A. Jun. 20, 2000;97(13):7196-201. This reference is herein incorporated by reference. Also, Alberts et al discusses the general nature of replication in bacteriophage T4 in “Studies on DNA replication in the bacteriophage T4 in vitro system” at Cold Spring Harb Symp Quant Biol. 1983;47 Pt 2:655-68. This reference is herein incorporated by reference.

In certain other embodiments of the present invention, the quantitative or qualitative isothermal screening and detection of one or more target nucleic acids of interest is performed in the presence of a helicase and a helicase accessory factor. The DNA helicase and the DNA helicase accessory factor can be derived from a eukaryotic or prokaryotic system. For example, the DNA helicase and the DNA helicase accessory factor can be from a bacterial system such as E. coli. or a bacteriophage system such as bacteriophage T4. For example, one DNA helicase/accessory factor pair is the bacteriophage T4 gene product 41 protein and its accessory factor gene product 59 protein. Jones et al discusses the gene product 59 protein in “Bacteriophage T4 gene 41 helicase and gene 59 helicase-loading protein: a versatile couple with roles in replication and recombination” at Proc Natl Acad Sci U S A. Jul. 17, 2001;98(15):8312-8. This reference is herein incorporated by reference.

In still other embodiments of the present invention, the quantitative or qualitative isothermal screening and detection of one or more target nucleic acids of interest is performed in the presence of a primosome. As used herein a primosome is a term that generally characterizes a complex comprising a DNA helicase and an RNA polymerase usually referred to as a primase. The primosome is active in synthesizing RNA primers on the lagging strand of a replication fork for the initiation of Okazaki fragment synthesis during coordinated leading- and lagging strand synthesis. Primases can be derived from a prokaryote or a eukaryote. For example, the primase can be from a bacterium such as E. coli., a bacteriophage such as bacteriophage T4 or bacteriophage T7, a yeast, or a human. One exemplar primase is the bacteriophage T4 gene product 61 protein, and derivatives or mutants thereof.

The phrase “amplification reaction reagents” as used herein includes but is not limited to reagents which are well known for their use in nucleic acid amplification reactions and may include but are not limited to: a single or multiple reagent, reagents, enzyme or enzymes separately or individually having reverse transcriptase and/or polymerase activity, strand transferase activity, or exonuclease activity; enzyme cofactors such as magnesium or manganese; salts; nicotinamide adenine dinucleotide (NAD); and deoxynucleoside triphosphates (dNTPs) such as, for example, deoxyadenosine triphosphate, deoxyguanosine triphosphate, deoxycytodine triphosphate and thymidine triphosphate. Other reagents include molecular crowding agents, including but not limited to polyethylene glycol PEG 8000. The exact amplification reagents employed are largely a matter of choice for one skilled in the art based upon the particular amplification reaction employed. For example, it is known in the art that volume occuping agents, or molecular crowding agents, inhance the activity or function of strand transferases, polymerases, and their accessory factors. The following references are herein incorporated by reference: (1) “Enhancement of recA Protein-promoted DNA Strand Exchange Activity by Volume occupying agents” at J Biol Chem. May 5, 1992;267(13):9307-14; (2) “Stimulation of the processivity of the DNA polymerase of bacteriophage T4 by the polymerase accessory proteins” at J Biol Chem. Jan. 25, 1999;266(3):1830-40; (3) “Macromolecular crowding”: thermodynamic consequences for protein-protein interactions within the T4 DNA replication complex: The role of ATP hydrolysis”; (4) “Macromolecular crowding”: thermodynamic consequences for protein-protein interactions within the T4 DNA replication complex” at J Biol Chem. Sep. 5, 1990;265(25):15160-7; (5) “Assembly of a functional replication complex without ATP hydrolysis: a direct interaction of bacteriophage T4 gp45 with T4 DNA polymerase” at Proc Natl Acad Sci U S A. Apr. 15, 1993;90(8):3211-5; and (6) “A coupled complex of T4 DNA replication helicase (gp41) and polymerase (gp43) can perform rapid and processive DNA strand-displacement synthesis” at Proc Natl Acad Sci U S A. Dec. 10, 1996;93(25): 14456-61.

### Target Nucleic Acids

Nucleic acid sequences that can be used in combination with the strand transferase dependent isothermal DNA amplification system described herein to specifically and sensitively detect a CK 19 target sequence have been decribed in U.S. Pat. No. 6,203,992 “Nucleic acid primers and probes for detecting tumor cells.” This reference is herein incorporated by reference. In particular, primers sequences disclosed include those in U.S. Pat. No. 6,203,992 designated SEQ ID NO 2 and SEQ ID NO 3. Sequences identified as SEQ ID NO 4, SEQ ID NO 5, SEQ ID NO 6, and SEQ ID NO 7, SEQ ID NO 10, were employed as probes for detecting the amplification product produced by SEQ. ID. NOs. 2 and 3. Combinations of the above sequences can be provided in kits having materials and reagents to perform the strand transferase dependent isothermal nucleic acid amplififcation described herein to detect a CK 19 target sequence in peripheral blood. The CK 19 target sequence was also desclosed, designated therein as SEQ. ID. NO. 1, and can be amplified by forming a reaction mixture comprising nucleic acid amplification reagents, a test sample containing a CK 19 target sequence, and a primer set containing SEQ ID NOs. 2 and 3. Following amplification, the amplified target sequence can be detected. For example, any probe or any combination of the probes designated SEQ ID NOs. 4, 5, 6, and 7 can be employed to hybridize to the amplified target sequence to form a probe/amplification product hybrid which can then be detected using microparticle capture techniques. Hence, the primers or probes can be labeled to capture and detect the amplified target sequence and therefore indicate the presence of the target sequence in the test sample.

The primer and probe sequences used in the present invention in combination with the strand transferase dependent isothermal DNA amplification desclosed and discussed herein, may comprise deoxyribonucleic acid (DNA), ribonucleic acid (RNA) or nucleic acid analogs such as uncharged nucleic acid analogs including but not limited to peptide nucleic acids (PNAs) which are disclosed in International Patent Application WO 92/20702 or morpholino analogs which are described in U.S. Pat. Nos. 5,185,444, 5,034,506, and 5,142,047 all of which are herein incorporated by reference. Such sequences can routinely be synthesized using a variety of techniques currently available. For example, a sequence of DNA can be synthesized using conventional nucleotide phosphoramidite chemistry and the instruments available from Applied Biosystems, Inc, (Foster City, Calif.); DuPont, (Wilmington, Del.); or Milligen, (Bedford, Mass.). Similarly, and when desirable, the sequences can be labeled using methodologies well known in the art such as described in U.S. Pat. Nos. 5,464,746; 5,424,414; and 4,948,882 all of which are herein incorporated by reference. It will be understood, however, that the sequences employed as primers should at least comprise DNA at the 3′ end of the sequence and preferably are completely comprised of DNA.

A “target sequence” as used herein means a nucleic acid sequence that is detected, amplified, both amplified and detected or otherwise is complementary to one of the sequences herein provided. While the term target sequence is sometimes referred to as single stranded, those skilled in the art will recognize that the target sequence may actually be double stranded.

The term “test sample” as used herein, means anything suspected of containing the target sequence. The test sample can be derived from any biological source, such as for example, blood, bronchial alveolar lavage, saliva, throat swabs, ocular lens fluid, cerebral spinal fluid, sweat, sputa, urine, milk, ascites fluid, mucous, synovial fluid, peritoneal fluid, amniotic fluid, tissues such as heart tissue and the like, or fermentation broths, cell cultures, chemical reaction mixtures and the like. The test sample can be used (i) directly as obtained from the source or (ii) following a pre-treatment to modify the character of the sample. Thus, the test sample can be pre-treated prior to use by, for example, preparing plasma from blood, disrupting cells, preparing liquids from solid materials, diluting viscous fluids, filtering liquids, distilling liquids, concentrating liquids, inactivating interfering components, adding reagents, purifying nucleic acids, and the like. Most typically, the test sample will be peripheral blood.

SEQ. ID. NOs. 2 and 3 can be used as amplification primers according to amplification procedures described herein. It will be understood by those skilled in the art that in the event that the target sequence is RNA, a reverse transcription step should be included in the amplification of the target sequence. Enzymes having reverse transcriptase activity, such as Rt TH, are well known for activity capable of synthesizing a DNA sequence from an RNA template. Reverse transcription PCR (RT PCR) is well known in the art and described in U.S. Pat. Nos. 5,310,652 and 5,322,770 which are herein incorporated by reference.

Thus, amplification methods of the present invention generally comprise the steps of (a) forming a reaction mixture comprising nucleic acid amplification reagents, SEQ. ID. NOS. 2 and 3, and a test sample suspected of containing a target sequence; and (b) subjecting the mixture to amplification conditions to generate at least one copy of a nucleic acid sequence complementary to the target sequence. It will be understood that step (b) of the above method can be repeated several times by thermal cycling the reaction mixture as is well known in the art.

As stated above, the reaction mixture comprises “nucleic acid amplification reagents” that include reagents which are well known and may include, but are not limited to, an enzyme having polymerase activity (and, as necessary, reverse transcriptase activity), enzyme cofactors such as magnesium or manganese; salts; nicotinamide adenine dinucleotide (NAD); and deoxynucleotide triphosphates (dNTPs) such as for example deoxyadenine triphosphate, deoxyguanine triphosphate, deoxycytosine triphosphate and deoxythymine triphosphate.

The amplification product produced as above can be detected during or subsequently to the amplification of the CK-19 target sequence. Methods for detecting the amplification of a target sequence during amplification are described in U.S. Pat. No. 5,210,015 that is herein incorporated by reference. Gel electrophoresis can be employed to detect the products of an amplification reaction after its completion. Preferably, however, amplification products are separated from other reaction components and detected using microparticles and labeled probes. Hence, methods for detecting the amplified CK-19 target sequence include the steps of (a) hybridizing at least one hybridization probe to the nucleic acid sequence complementary to the target sequence, so as to form a hybrid comprising the probe and the nucleic acid sequence complementary to the target sequence; and (b) detecting the hybrid as an indication of the presence of the presence of the target seuquence in the test sample.

Hybrids formed as above can be detected using microparticles and labels that can be used to separate and detect such hybrids. Preferably, detection is performed according to the protocols used by the commercially available Abbott LCx.RTM. instrumentation (Abbott Laboratories; Abbott Park, Ill.).

The term “label” as used herein means a molecule or moiety having a property or characteristic which is capable of detection. A label can be directly detectable, as with, for example, radioisotopes, fluorophores, chemiluminophores, enzymes, colloidal particles, fluorescent microparticles and the like; or a label may be indirectly detectable, as with, for example, specific binding members. It will be understood that directly detectable labels may require additional components such as, for example, substrates, triggering reagents, light, and the like to enable detection of the label. When indirectly detectable labels are used, they are typically used in combination with a “conjugate”. A conjugate is typically a specific binding member which has been attached or coupled to a directly detectable label. Coupling chemistries for synthesizing a conjugate are well known in the art and can include, for example, any chemical means and/or physical means that does not destroy the specific binding property of the specific binding member or the detectable property of the label. As used herein, “specific binding member” means a member of a binding pair, i.e., two different molecules where one of the molecules through, for example, chemical or physical means specifically binds to the other molecule. In addition to antigen and antibody specific binding pairs, other specific binding pairs include, but are not intended to be limited to, avidin and biotin; haptens and antibodies specific for haptens; complementary nucleotide sequences; enzyme cofactors or substrates and enzymes; and the like.

A “microparticle”, refers to any material which is insoluble, or can be made insoluble by a subsequent reaction and is in a particulate form. Thus, microparticles can be latex, plastic, derivatized plastic, magnetic or non-magnetic metal, glass, silicon or the like. A vast array of microparticle configurations are also well known and include, but are not intended to be limited to, beads, shavings, grains, or other particles, well known to those skilled in the art. Microparticles according to the invention preferably are between 0.1 .mu.M and 1 .mu.M in size and more preferably between 0.3 .mu.M and 0.6 mu.M in size.

According to one embodiment, hybrids can be detected by incorporating labels in the primer and/or probe sequences to facilitate detection. Hence, first and second specific binding members attached to the primers and probes can be employed to immobilize the hybrids to microparticles and detect the presence of the microparticles with the assistance of a conjugate.

According to another embodiment, a combination of specific binding members and directly detectable labels can be employed to detect hybrids. For example, specific binding members can be introduced in the hybrids using primers labeled with specific binding members. A directly detectable label can be incorporated into the hybrids using a probe that has been labeled with a directly detectable label. Hence, hybrids can be immobilized to a microparticle using the specific binding member and directly detected by virtue of the label on the probe. It will be understood that other detection configurations are a matter of choice for those skilled in the art.

According to one embodiment, the strand transferase dependent isothermal amplification reactions disclosed herein can be employed to detect the CK19 target sequence. Briefly, the reagents employed in the preferred method comprise at least one amplification primer and at least one internal hybridization probe, as well amplification reagents for performing the strand transferase dependent amplification described herein.

The primer sequence is employed to prime extension of a copy of a target sequence (or its complement) and is labeled with either a capture label or a detection label. The probe sequence is used to hybridize with the sequence generated by the primer sequence, and typically hybridizes with a sequence that does not include the primer sequence. Similarly to the primer sequence, the probe sequence is also labeled with either a capture label or a detection label with the caveat that when the primer is labeled with a capture label the probe is labeled with a detection label and vice versa. Detection labels have the same definition as “labels” previously defined and “capture labels” are typically used to separate extension products, and probes associated with any such products, from other amplification reactants. Specific binding members (as previously defined) are well suited for this purpose. Also, probes used according to this method are preferably blocked at their 3′ ends so that they are not extended under hybridization conditions. Methods for preventing extension of a probe are well known and are a matter of choice for one skilled in the art. Typically, adding a phosphate group to the 3′ end of the probe will suffice for purposes of blocking extension of the probe. According to the certain embodiments the probe initially is part of the reaction mixture.

As previously mentioned, the present invention provides reagents, methods, and kits for amplifying and detecting a CK-19 target sequence in a test sample. In particular, SEQ. ID. Nos. 2 and 3 can be employed as amplification primers to amplify the CK 19 target sequence designated SEQ. ID. NO. 1 in U.S. Pat. No. 6,203,992. These primers can be used with the strand transferase dependent isothermal DNA amplification stem described herein to specifically and sensitively produce an amplification product that is amenable to microparticle capture and detection techniques. Probe sequences, having SEQ. ID. Nos. 4 through 7, as disclosed in U.S. Pat. No. 6,203,992 can be employed to insure specificity and detect the amplification product.

What is claimed is:

1. A method for detecting the presence of a tumor cell in a test sample comprising contacting a test sample with a strand transferase, at least one polymerase, and at least one single stranded nucleic acid having complementarity to tumor cell nucleic acid.

2. The method of claim 1 wherein said strand transferase is derived from a prokaryotic.

3. The method of claim 1 wherein said strand transferase is the uvsX strand transferase derived from the bacteriophage T4.

4. The polymerase of claim 1 wherein said polymerase is derived from a prokaryotic.

5. The polymerase of claim 1 wherein said polymerase is the gp43 polymerase derived from the bacteriophage T4.
